(19) United States
(12) Patent Application Publication
(10) Pub. No.: US 2020/0038708 A1
(43) Pub. Date: Feb. 6, 2020

(54) SYSTEM AND METHOD FOR OPTIMIZING DIAPHRAGMATIC BREATHING

(71) Applicants: Dwight CHEU, Albany, NY (US); Michael DICESARE, Albany, NY (US)

(72) Inventors: Dwight CHEU, Albany, NY (US); Michael DICESARE, Albany, NY (US)

(21) Appl. No.: 16/528,519

(22) Filed: Jul. 31, 2019

Related U.S. Application Data
(60) Provisional application No. 62/713,381, filed on Aug. 1, 2018.

(51) Int. Cl. A63B 23/18; A63B 24/00; A61B 5/00; A63B 71/06; G16H 50/20; G06N 20/20

(52) U.S. Cl. CPC A63B 23/185; A63B 24/0075; A61B 5/6898; A61B 5/682; A61B 5/6823; A63B 71/0622; A61B 5/091; G06N 20/20; A63B 2230/40; A61B 2562/0219; A63B 2225/20; A63B 2024/0068; G16H 50/20

(57) ABSTRACT

A system to optimize diaphragmatic breathing is disclosed. The system has a first sensor to measure breathing movement of a user's abdomen and output a signal related to the movement of the user's abdomen, a second sensor to measure breathing movement of the user's chest and output a signal related to the movement of the user's chest; and a control device communicatively coupled with the first sensor and the second sensor. The control device has one or more processors, a memory comprising set of program modules executable by one or more processors. an assessment module for receiving the signal from the first sensor and second sensor and converting the signals to a data input, and for comparing the data input to a predetermined data range representative of proper diaphragmatic breathing for the user and a communication interface for providing feedback based on the assessment modules comparison of the data input and the predetermined range so as to optimize the user's diaphragmatic breathing. A method for optimizing diaphragmatic breathing is also disclosed.

### CROSS-REFERENCE TO RELATED APPLICATIONS

This application claims the benefit of U.S. Provisional Application Ser. No. 62/713,381 entitled System and Method for Optimizing Diaphragmatic Breathing filed on Aug. 1, 2018, the entire contents of which are incorporated herein by reference for all purposes.

### FIELD OF THE INVENTION

The present invention relates generally to systems and methods for performing diaphragmatic breathing. More particularly, the present invention relates to certain new and useful advances in the field of healthcare and pain management to train users how to properly perform diaphragmatic breathing, where gamification is used to create an interactive learning experience with real-time feedback to create a more effective and efficient training process; references being had to the drawings accompanying and forming a part of the same.

### BACKGROUND

The diaphragm is a large, dome-shaped muscle located at the base of a human's lungs between the thoracic cavity and abdominal cavity that is often described as being the most efficient muscle of breathing. In essence, your abdominal muscles aid in moving the diaphragm and provide you with the power necessary to empty your lungs. However, there are many reasons and more particularly, diseases, that can prevent your diaphragm from operating properly.

The number of opioid overdose deaths in the United States continues to rise, with approximately 130 Americans dying from opioid overdoses every day (NIDA, 2018). One of the major contributing factors to this epidemic is the increase in prescriptions of natural and synthetic opioids for pain relief and pain management that began in the late 1990s (CDC 2017; Kolodny et al, 2015). Though opioids are prescribed to treat a variety of conditions, a recent analysis of opioid related deaths of people on Medicare under age 65 found that 61.5% of decedents were diagnosed with a chronic pain condition in the year preceding their death, with 59.3% of those diagnoses being for back pain (Olfson et al, 2017). A systematic review of studies on opioid use for the treatment of back pain found that 36-56% of individuals prescribed opioids for chronic back pain showed evidence of long-term substance use disorders (Martell et al, 2007). One of the recognized methods for reducing dependency on opioids for back pain treatment is to prescribe physical therapy as a first course of treatment. When patients with a diagnosis of lower back pain saw a physical therapist (PT) first, they were 89.4% less likely to be prescribed opioids to treat their pain (Frogner et al, 2018). Despite the benefits of implementing non-pharmacological approaches first, pharmacological approaches are still overutilized, with opioids being prescribed for almost 50% of lower back pain cases, compared to only 12% of cases being prescribed to PT (Salt et al, 2016).

Diaphragmatic breathing (also known as deep breathing, abdominal breathing, or paced respiration) is a breathing technique that teaches patients to contract their diaphragm by watching their stomach expand and keeping the chest still as they breath. The technique is one form of therapy that can be prescribed to individuals undergoing physical therapy for a variety of neck, back, shoulder and pelvic floor diagnoses. Musculoskeletal imbalances are considered to be both an underlying cause, and a resulting symptom of dysfunctional breathing, or breathing pattern disorders (BPDs) as the mechanics of breathing are key to both spinal stability and posture (CliftonSmith & Rowley, 2011). The symptoms of patients with BPD's vary widely, but can include mental factors such as anxiety and depression as well as physical symptoms like chronic back and neck pain and fatigue, and neck and shoulder problems (Courtney, 2009; Perry & Halford, 2004; CliftonSmith & Rowley, 2011). There is a growing recognition that breathing retraining and breath therapy can be used to address a variety of BPD's, reducing the associated mental and physical symptoms (CliftonSmith & Rowley, 2011). Breath therapy can also be a key component in pain management (Mehling, 2005; Busch et al. 2012) and anxiety reduction (Chen et al, 2017). In a study of chronic lower back pain, patients prescribed to a breath therapy routine that included diaphragmatic breathing showed significant decreases in pain levels and improvements in function as a result of this therapy (Mehling et al, 2005).

To perform the diaphragmatic breathing technique, patients are instructed to lie on their back with knees elevated, placing one hand on their belly and one on their chest. They are instructed to take a deep breath in slowly through the nose, feeling their stomach rise while keeping their chest still, and then to exhale through pursed lips, feeling the hand on their stomach fall (CDC, 2014; Cleveland Clinic, 2017). Patients are typically guided through the technique by a physical therapist (PT), or by a video or pictorial description of the procedure. Feedback is achieved by feeling your hands rise and fall (or in one video, participants are instructed to place one shoe on their stomach and one on their chest). While video or pictorial instructions can be useful in helping the patient remember the steps of the procedure when they are at home, this does not provide them with any feedback or real coaching on technique performance.

In addition to pain management, respiratory diseases such as chronic obstructive pulmonary diseases (COPD) is one such disease, where air often becomes trapped in the lungs and is pushed down onto the diaphragm. As a result of this, extra stress is then placed on the neck and chest muscles to ensure proper breathing is maintained. Affected muscles include the scalene (in the neck); pectoralis major and minor (in the chest); serratus anterior (wraps around the ribcage on the underside of the arms); and serratus posterior superior and inferior (in the back). And while these muscles are meant to assist in proper breathing, they are not meant to supplement or otherwise replace the purpose of the diaphragm. Often times, this leaves the diaphragm weak and flattened, and operating in a state that is less efficient than what is desired.

This breathing technique focuses on strengthening the transverse abdominal muscles which in turn provides the basis of support of the lower spine. Diaphragmatic breathing also increases the strength of this muscle group and also helps to training these muscles to activate in the right sequence as shown in multiple studies.

A healthcare provider often teaches patients how to properly perform diaphragmatic breathing by placing one hand on the patient's abdomen and another on the patient's chest. The healthcare provider then guides the patient through the breathing routine, instructing the patient to expand their abdomen while trying to keep their chest as still as possible. This is a very intensive, resource-driven way to learn the diaphragmatic breathing technique since it requires in-person coaching by a trained health care professional.

In addition to in person teaching by a health practitioner such as a physical therapist, other examples of methods to teach breathing techniques include: (i) placing objects such as a shoe on the subject's chest and one on the subject's abdomen as a means to monitor movement and breathing patterns; (ii) using devices such as Respitrace™ to monitor breathing patterns including the detection of paradoxical breathing and abnormal breathing patterns in sleep through the use of a system of winding coils and elastic bands that strap onto a user's chest and abdomen and attach to a central processing unit via a wired connection; (iii) using devices such as Spire™, which is a single wearable sensor that continuously monitors a user's respiratory patterns throughout the day and generates reports and feedback; and (iv) applications for smartphones that walk users through breathing routines for meditation and the treatment of anxiety.

Another existing system is taught in US Patent Application Publication No. US20150342518 to Persidsky et al., in which a system and method to monitor, guide, and evaluate breathing is disclosed. The system monitors user's breathing with respect to user definable breathing patterns, sequences, and preexisting breathing exercises, utilizing posture and diaphragm sensor signals and a method to process thereof, composed of hardware and software components. The application describes a system which monitors the output signals of sensors as part of a breath training device worn by a user for measuring the state of a user's posture and diaphragm to derive a filtered breath signal.

Further existing systems such as that described in US20160038083 of Ding et al., discloses a garment including integrated sensor components and feedback components. The garment is used for measuring one or more parameters of a wearer includes a base material configured to be worn by a wearer and a sensing component. The sensing component is integrated into a first location of the base material corresponding to a predetermined region of the wearer.

However, these systems and methods suffer from various drawbacks as it relates specifically to the types and locations of the sensors as it relates to the body, training users to breath diaphragmatically in a manner that treats certain conditions, and further, in a manner that provides feedback required to make the devices effective.

As such, there is a need for a system and method that can teach and incentives individuals or diaphragmatically breath properly and relieve pain or other conditions.

### SUMMARY OF THE INVENTION

The following summary of the invention is provided in order to provide a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented below.

To achieve the foregoing and other aspects and in accordance with the purpose of the invention, a system and method for teaching and performing diaphragmatic breathing is presented.

Accordingly, a new and improved system and method that serves multiple purposes and is convenient and easy to use is provided.

Further, a diaphragmatic training system and method as an alternative to opioids for pain management is provided.

Further, a new and improved system and method to increase the pain tolerance and adherence of the users to perform prescribed diaphragmatic breathing exercises and thereby strengthening lower back muscles is provided.

Further, a new and improve system and method that is an alternative or augmentation to in person physical therapy visits is provided.

Further, a new and improved system and method utilizing gamification that increases the likelihood of a user meeting therapy goals and thus creating an overall better and more effective therapy experience is provided.

Further, a new and improved system to perform diaphragmatic breathing exercises is provided. In addition, rather than using traditional in-person exercises led by a physical therapist, a biofeedback sensor system is used to integrate a user's real-time breathing pattern while using a video game to control the input. Creating a system around gamification is important because it creates a fun and engaging low cost alternative to the traditional techniques normally performed by a physical therapist at a health care provider's office.

Further, a system that can accurately measure and monitor respiratory exercise and provide user feedback is provided.

Further, a system that optimizes the diaphragmatic breathing training techniques that that is specific to a particular diagnosis, such as pain management or COPD for example is provided.

Further, the system utilizes machine learning so that the system improves over time with respect to each individual's needs, and also, to optimize the approach on a diagnosis specific level.

In exemplary embodiments, a system and method for performing diaphragmatic breathing is presented. The system comprises a first sensor to measure breathing movement of a user's abdomen and output a signal related to the movement of the user's abdomen; a second sensor to measure breathing movement of the user's chest and output a signal related to the movement of the user's chest; and a control device communicatively coupled with the first sensor and the second sensor, wherein the control device comprises: one or more processors; a memory comprising set of program modules executable by one or more processors comprising: an assessment module for receiving the signal from the first sensor and second sensor and converting the signals to a data input, and for comparing the data input to a predetermined data range representative of proper diaphragmatic breathing for the user; and a communication interface for providing feedback based on the assessment modules comparison of the data input and the predetermined range so as to optimize the user's diaphragmatic breathing.

In one embodiment, the control device comprises one or more user interface devices to provide feedback. In one embodiment, the user interface devices include at least one of, but not limited to, displays, monitors, keyboards, and pointing devices. In one embodiment, the control device is configured to provide vibratory feedback via the first sensor to adjust breathing movement of the abdomen. In one embodiment, the control device is further configured to provide vibratory feedback via the second sensor to adjust breathing movement of the chest. In one embodiment, the control device is at least any one of, but not limited to, a computing device, a mobile device, a wearable device, or a wearable digital headset device.

In one embodiment, the control device further comprises one or more processors and a memory. The one or more processors are implemented with one or more machine learning algorithms. In one embodiment, the memory comprises a set of program modules that executable by the one or more processors. In one embodiment, the control device further comprises an interactive gaming module, an assessment module, and an optimization engine. In one embodiment, the interactive gaming module in communication with a gaming platform comprising one or more therapy games, configured to enable the user to control one or more therapy games using diaphragmatic breathing. In one embodiment, the assessment module in communication with an assessment database, configured to store performance data related to each therapy game played by the user. In one embodiment, the optimization engine in communication with the assessment module configured to make real time adjustments to the one or more therapy games based on the performance data to optimize diaphragmatic breathing.

In one embodiment, the system further comprises a third sensor. The third sensor is configured to measure an amount of air exhaled/inhaled by the user's mouth, and should the user be breathing in/out of their mouth, communicate with the control device to alert the user they are incorrectly performing the breathing technique. Furthermore, a fourth sensor in the form of a nasal sensor may be employed to measure air flow from the nasal passages of the user.

In one embodiment, the first sensor is disposed at the user's abdomen, the second sensor is disposed at the user's chest, and the third sensor is disposed at the user's mouth. In one embodiment, the third sensor may be, but not limited to, a mouth sensor, a mouthpiece sensor, and a Bluetooth mouthpiece. The third sensor is configured to detect an upward movement of the first sensor via absence of airflow in the mouthpiece, and detect a downward movement of the first sensor via air exiting through the mouthpiece.

In exemplary embodiments, a method to optimize diaphragmatic breathing, is provide. The method comprises locating a first sensor proximate a user's abdomen; locating a second sensor proximate a user's chest; capturing, using the first and second sensor, movement of the chest and abdomen during user breathing; outputting a signal based on the movement of the chest and abdomen to a control device via a communication protocol; receiving the signal from the first sensor and second sensor and converting the signals to a data input via an assessment module in communication with a processor; comparing, using a processor, the data input to a predetermined data range representative of proper diaphragmatic breathing for the user; and providing feedback based on the assessment modules comparison of the data input and the predetermined range so as to optimize the user's diaphragmatic breathing.

The system comprises a first sensor, a second sensor, and a control device. In one embodiment, the control device is a gaming server that enables a group of users having a similar ailment in widely distributed geographical locations to play the same game within the same game environment at the same time. In one embodiment, a medical professional may group a number of users based on certain scores, and other various metadata such as, but not limited to, user age, gender, weight, and the like.

Other features, advantages, and aspects of the present invention will become more apparent and be more readily understood from the following detailed description, which should be read in conjunction with the accompanying drawings.

### BRIEF DESCRIPTION OF THE DRAWINGS

FIG. 1 is a system diagram of an exemplary diaphragmatic breathing system in accordance with one embodiment;

FIG. 2 is a perspective view and component diagram illustrating the internal components of the sensors used in the diaphragmatic breathing system in accordance with one embodiment;

FIG. 3 is a perspective view diagram of the sensors being used in accordance with one embodiment;

FIG. 4a is exemplary accelerometer sensor feedback graph used with the diaphragmatic breathing system in accordance with one embodiment;

FIG. 4b is another exemplary accelerometer sensor feedback graph used with the diaphragmatic breathing system in accordance with one embodiment;

FIG. 5 is an exemplary gameplay flow for use with the diaphragmatic breathing system in accordance with one embodiment;

FIG. 6 is an exemplary gameplay flow for use with the diaphragmatic breathing system in accordance with one embodiment;

FIG. 7 is an exemplary embodiment of a machine learning processes for optimizing diaphragmatic breathing routines for a user;

FIG. 8 is front view of an optional embodiment of a sensor strip having vibratory elements being used in an embodiment;

FIG. 9 is a step-wise method diagram for a method for optimizing diaphragmatic breathing;

FIG. 10 is a perspective view diagram of a third sensor being used together with a chest and abdominal sensor in accordance with one embodiment;

FIG. 11 is system diagram of an exemplary diaphragmatic breathing system in accordance with one embodiment;

FIG. 12 is an exemplary game flow and VR system for use with the diaphragmatic breathing system in accordance with one embodiment;

FIG. 13 is a perspective view diagram of a smartphone being used as the sensing apparatus in accordance with one embodiment.

### DETAILED DESCRIPTION OF THE INVENTION

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described are shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be also understood to refer to functional equivalents of such structures. The present invention will now be described in detail with reference to embodiments thereof as illustrated in the accompanying drawings.

Specific configurations and arrangements of the invention, discussed above regarding the accompanying drawing, are for illustrative purposes only. Other configurations and arrangements that are within the purview of a skilled artisan can be made, used, or sold without departing from the spirit and scope of the invention. For example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures.

As used herein, the term “user” shall mean any individual who uses the system to perform diaphragmatic breathing or to otherwise aide their breathing issues. More specifically, a user will either be a patient who has been prescribed breathing exercises or an individual who is seeking an in-home therapy treatment to help with diaphragm related health issues.

As used herein, the term “optimize” shall mean any change in diaphragmatic breathing that may considered helpful or useful to a user based on their condition or treatment program.

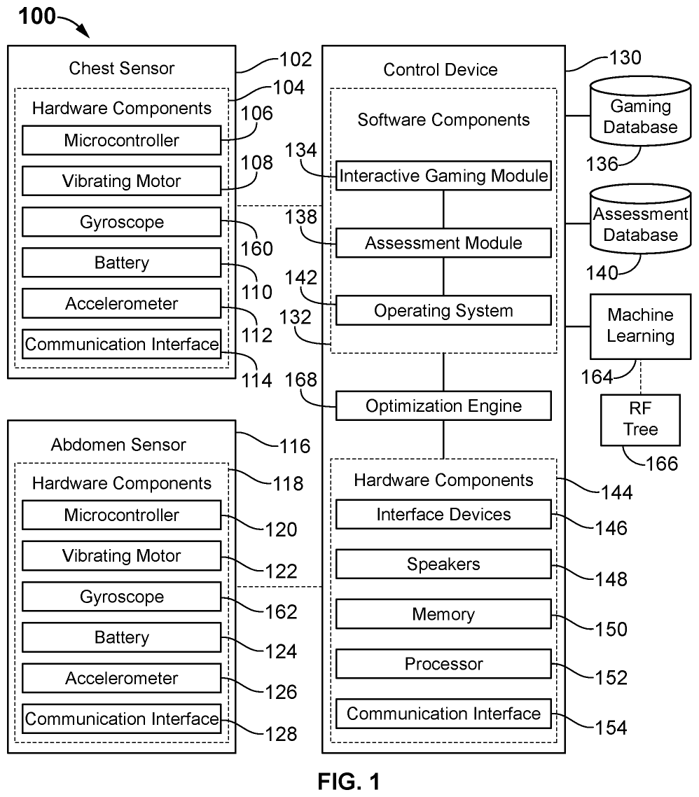

Referring now to FIG. 1, a diagram of an exemplary diaphragmatic breathing system in accordance with one embodiment is shown generally at reference numeral 100. The embodiment 100 shows a diaphragmatic system for use in a therapy environment in which a first sensor or a chest sensor 102 and a second sensor or an abdomen sensor 116 act together with the control device 130 to train a patient or user on the best technique for proper diaphragmatic breathing based upon a medical condition input by an operator (e.g., medical professional).

The embodiment 100 illustrates the functional components of a system. In some embodiments, the functional component may be a hardware component, a software component, or a combination of hardware and software. Some of the components may be application level software, while other components may be operating system level components. In some cases, the connection of one component to another may be a close connection where two or more components are operating on a single hardware platform. In other cases, the connection may be made over network connections spanning long distances and a plurality of nodes. Each embodiment may use different hardware, software, and interconnection architectures to achieve the described functions.

Still referring to FIG. 1, the chest sensor 102 may also comprise a plurality of hardware components 104. In the current embodiment, the hardware components 104 may comprise a microcontroller 106, a vibrating motor 108, a power source 110, an accelerometer 112, a gyroscope 160, and a communication interface 114. The communication interface 114 may comprise hardwired and wireless interfaces through which the chest sensor 102 may communicate with other devices, a network, and a plurality of servers. In the current embodiment, the communication interface 114 may comprise a Bluetooth® module that allows the chest sensor 102 to communicate wirelessly with the control device 130 and other system components at short range. In optional embodiments, the communication interface 114 may utilize other wireless technology or even wired technology depending on the usage including but not limited to, Wi-Fi, LTE, GSM/EGE. CDMA, WiMAX, NFC, etc.

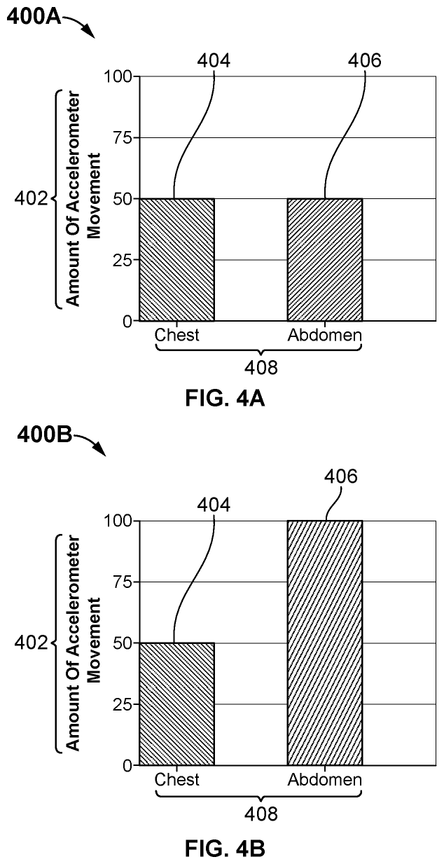

Still referring to FIG. 1, in the current embodiment the power source 110 may comprise a battery such as, but not limited to, a rechargeable lithium ion. The accelerometer 112 is configured to sense movement along the X, Y, and Z axis. The operation of the accelerometer 112 will be further discussed in regard to FIGS. 4-6. The gyroscope 160 is configured to sense a change in rotation which is unaffected by acceleration. In operation embodiments, a magnetometer may be used as well. The chest sensor 102 further comprises a microcontroller 106 that is used to control and operate the vibrating motors 108, the power source, for example, a battery 110, the accelerometer 112, and the communication interface 116. In one embodiment, the microcontroller 106 is a small computer on a single integrated circuit chip, and comprises one or more central processing units (CPUs) along with memory and programmable input/output peripherals. In optional embodiments, a different computing device may be used to operate the vibrating motors 108, the power source, for example, a battery 110, the accelerometer 112, and the communication interface 114.

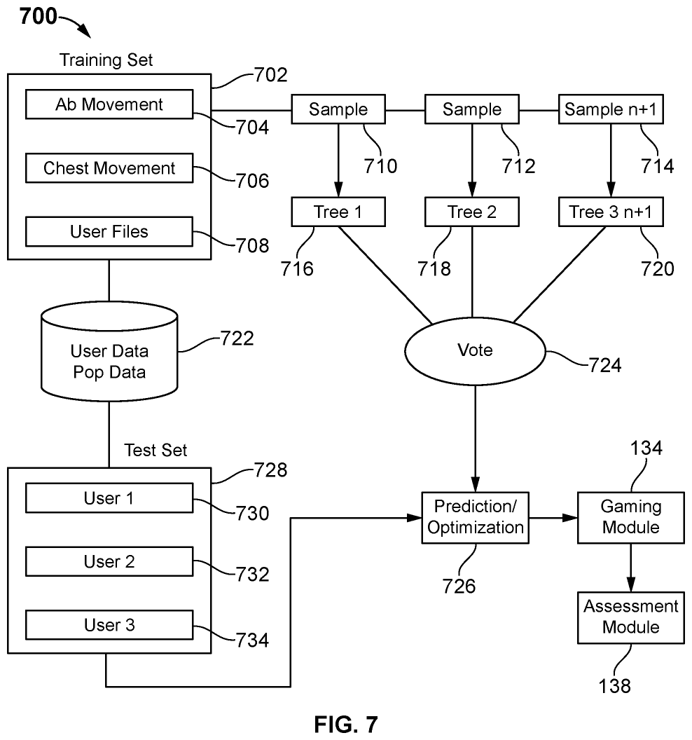

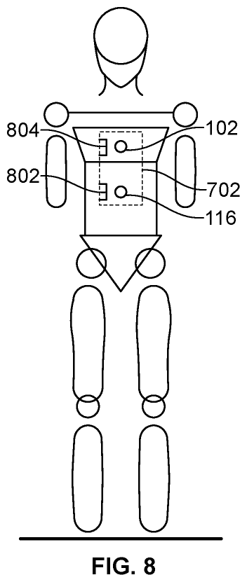

In operation, each of the hardware and software components of the chest sensor 102 are configured to assess breathing movements of the chest, and use the data outputs to plot a graph and further, use the data outputs as inputs to a machine learning algorithm discussed in greater detail with reference to FIGS. 7 and 8.

Still referring to FIG. 1, in addition to the chest sensor 102 of the system may also comprise an abdomen sensor 116. In embodiments, the chest sensor 102 and the abdomen sensor 116 may be referred to as a first sensor and a second sensor, respectively. The abdomen sensor 116 may comprise the same or similar components present in the chest sensor 102. More particularly, the abdomen sensor 116 may comprise certain hardware components 118, which comprise a microcontroller 120, a vibrating motor 122, a power source, for example, a battery 124, an accelerometer 126, a gyroscope 162, and a communication interface 128. The communication interface 128 may comprise hardwired and wireless interfaces through which the abdomen sensor 116 may communicate with other devices. In the current embodiment, the communication interface 128 may comprise a Bluetooth® module with an antenna that allows the abdomen sensor 116 to communicate wirelessly with the control device 130. In additional embodiments, the communication interface 128 may utilize other wireless technology or even wired technology depending on the exact usage, including but not limited to, Wi-Fi, LTE, GSM/EGE. CDMA, WiMAX, NFC, etc.

Still referring to FIG. 1, in the current embodiment the power source 124 is, but not limited to, a single replaceable lithium battery. In optional embodiments, the battery may be a rechargeable battery such as a lithium ion battery or may comprise more than one replaceable lithium battery. The power source 124 serves as the power source for the abdomen sensor 116. In the current embodiment, the accelerometer 126 is configured to sense movement along the X, Y, and Z axis. The gyroscope 162 is configured to sense a change in rotation which is unaffected by acceleration. In operation embodiments, a magnetometer may be used as well. In the current embodiment, the abdomen sensor 116 comprises a microcontroller 120 that is used to control and operate the vibrating motors 122, the power source, for example, a battery 124, the accelerometer 126, and the communication interface 128. In one embodiment, the microcontroller 120 is a small computer on a single integrated circuit chip, and comprises one or more central processing units (CPUs) along with memory and programmable input/output peripherals. In optional embodiments, a different computing device may be used to operate the vibrating motors 122, a power source, for example, a battery 124, the accelerometer 126, and the communication interface 128.

Still referring to FIG. 1, the chest sensor 102 and the abdomen sensor 116 are communicatively coupled to the control device 130. The chest sensor 102 and the abdomen sensor 116 are communicatively coupled to the control device 130 through the use of their communication interfaces (114 and 128), respectively, which may comprise a wireless Bluetooth® connection and antenna. In optional embodiments, the chest sensor 102 and the abdomen sensor 116 may be communicatively coupled to the control device 130 through the use of wired technology or another wireless technology such as, but not limited to, Wi-Fi, LTE, GSM/EGE. CDMA, WiMAX, NFC, etc. In one embodiment, the control device 130 may represent an architecture of a computing device.

In embodiments, the control device 130 is representative of a computing system having multiple modules. The control device 130 may also be a portable device, such as, but not limited to, a tablet, a laptop computer, a netbook computer, a personal digital assistant (PDA), a mobile telephone, or other mobile device, and other communication devices. In the current embodiment, the control device 130 is a portable device in the form of a tablet, but in optional embodiments the control device 130 may be a mobile phone, a laptop or a desktop computer. In optional embodiments, the control device 130 may be a wearable digital headset such as a virtual reality headset that allows the system 100 to be hands free, or an augmented reality headset that is hands free. In other words, the control device 130 may be any form factor that is usable by a user and capable of operating software components 132.

Still referring to FIG. 1, in the present embodiment the control device 130 may comprise hardware components 144 may comprise interface devices 146, speakers 148, a memory 150, a processor or a central processing unit 152, and a communication interface 154. In the current embodiment, the interface devices 146 may comprise, but not limited to, monitors, displays, keyboards, pointing devices, and any other type of user interface device. The random-access memory 150 may store executable code as well as data that may be immediately accessible to the processor 152. In the current embodiment, the random-access memory 150 is used to store any data needed to play the games. The speakers 148 are used to project sounds that are emitted from the control device 130.

Still referring to FIG. 1, the communication interface 154 may also comprise hardwired and wireless interfaces through which the control device 130 may communicate with other devices. In the current embodiment, the control device 130 will utilize the communication interface 154 to communicate with the chest sensor 102 and the abdomen sensor 116 via, but not limited to a wireless technology, for example, a Bluetooth® technology. The communication interface 154 also allows the control device 130 to communicate with certain other remote access devices, which may comprise any off-site device used by a physician or therapy provider to review results and set parameters for new therapy sessions.

Still referring to FIG. 1, in the present embodiment the control device 130 may also comprise software components 132. The software components 132 may be in the form of an application downloadable from a mobile/web app store, for example, or come preloaded into a hardware form factor. The software components 132 may comprise an operating system 142 on a non-transitory media on which various applications may execute, as well as an interactive gaming module 134 and an assessment module 138. The interactive gaming module 134 allows for the operation of any of the therapy games. The interactive gaming module 134 is in communication with a gaming database 136 which stores all of the therapy games. In the embodiments, the gaming database 136 may be updated via use of the communication interface 154, whereby the user can connect to a remote device such as a third-party storage device or to a virtual store where new games can be stored in the gaming database 136 and played by a user via the interactive gaming module 134.

Still referring to FIG. 1, the software components 132 may also comprise an assessment module 138, which is used to analyze and score a user's performance on any game played via the interactive gaming module 134, and which is coupled to an assessment database 140. After the performance of each gaming session, the scores are stored in the assessment database 140, and may be recalled by the interactive gaming module 134 to make user specific adjustments to a game to account for the user's therapy performance. The scores stored in the assessment database 140 may also be accessed by third parties such as therapy providers and doctors via use of the communication interface 154, who may use the assessments to modify the specific user's games in the gaming database 136 to maximize the effectiveness and efficiency of a particular therapy session, thus allowing for both in-person and remote therapy assessments. In one embodiment, the gaming database 136 and the assessment database 140 are likely contained within the control device 130, these databases may also be remote, say on a server computer in the same building or in the Internet or a cloud.

An optimization engine 168 is in communication with the control device 130 and the software components 132, specifically, the assessment module 138 and the assessment database 140. The optimization engine 168 collects all of the data regarding the user's performance via the assessment module 138. The optimization engine 168, via the machine learning module 164, is configured to make real time adjustments to the interactive games based on user performance, medical practitioner input, or both. Examples of machine learning that may be employed are, but not limited to, neural networks, convolution neural networks, Random Forest (RF Tree 166), which is discussed in greater detail with reference to FIG. 7, and the like.

In one embodiment, the control device 130 is a gaming server that enables a group of users having a similar ailment in widely distributed geographical locations to play the same game within the same game environment at the same time. In one embodiment, a medical professional could group a number of users based on certain scores, and other various metadata such as, but not limited to, user age, gender, weight, and the like.

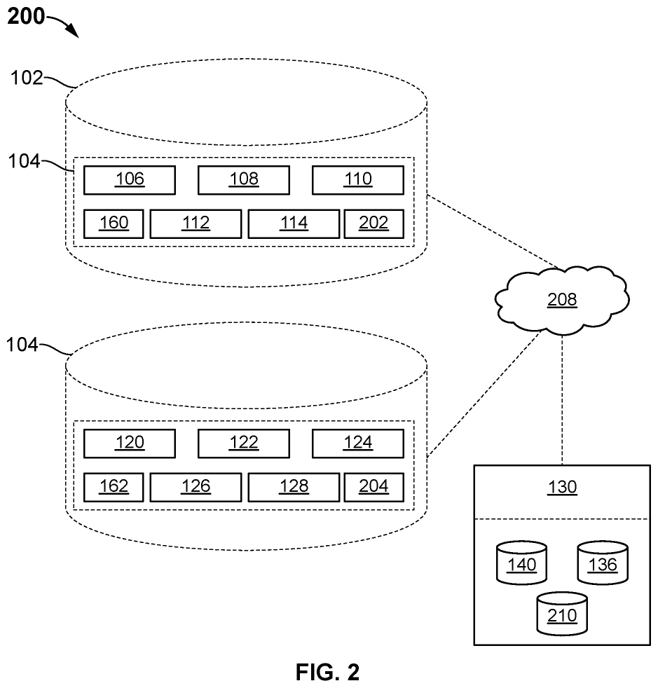

Referring now to FIG. 2, an exploded view illustrating the view illustrating the internal components of the sensors used in the diaphragmatic breathing system in accordance with one embodiment of the present system, is presented generally at 200. The chest sensor 102 comprises certain hardware components 104, which may comprise a microcontroller 106, a vibrating motor 108, a battery 110, an accelerometer 112, a gyroscope 160, and a communication interface 114. The abdomen sensor 116 comprises certain hardware components 118, which may comprise a microcontroller 120, a vibrating motor 122, a battery 124, an accelerometer 126, a gyroscope 162, and a communication interface 128. In optional embodiments, the chest sensor 102 and the abdomen sensor 116 may also comprise additional hardware components. The operation of the hardware components 104 for the chest sensor 102 and the hardware components 118 for the abdomen sensor 116 were previously discussed in detail with relation to FIG. 1. While accelerometers and gyroscopes are referred to herein, strain gauges and other types of sensors may be employed as well.

Referring still to FIG. 2, each sensor (102 and 116) is in communication with the network 208, which in turn relays information to the control device 130 and the databases (136 and 140). A plurality of servers 210 is in communication with the network 208 and the sensors (102 and 116), and configured to store and run plurality of machine learning programs for thousands of users to output training data sets that will be used by the optimization engine for user-specific game performance.

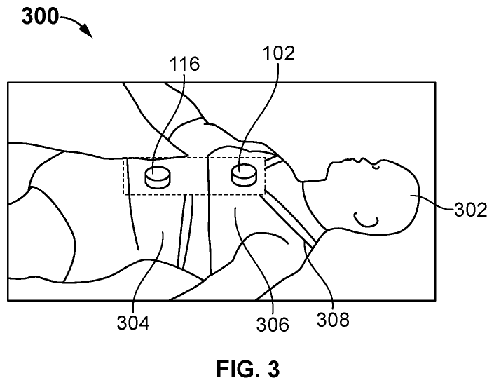

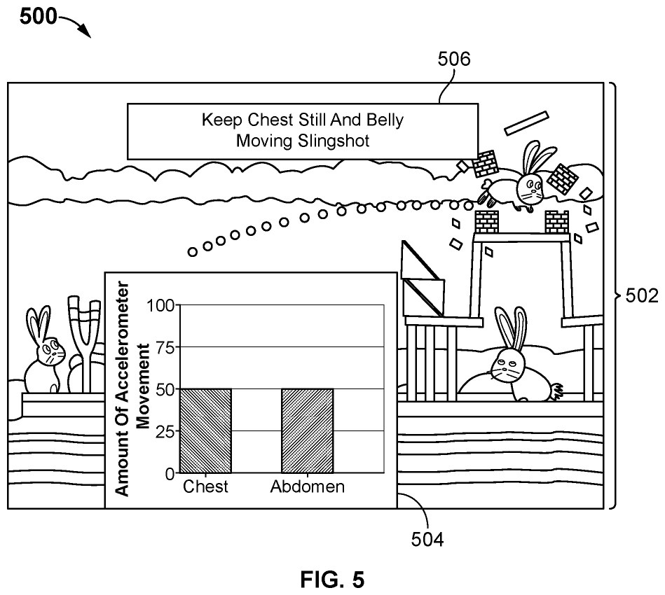

Referring now to FIG. 3, a perspective view diagram of the sensors being used in accordance with one embodiment of the present system is presented generally at 300. In this embodiment, a user 302 is scene in a position to accept the abdomen sensor 116 and the chest sensor 102. The abdomen sensor 102 is placed on the center of the abdomen 304 of the user 302. The chest sensor 102 is placed on the center of the chest 306 of the user 302. In the current embodiment, the abdomen sensor 116 and the chest sensor 102 are temporarily attached on top of the clothes of a user 302 and are not placed directly in contact with the skin of the user 302. In optional embodiments, the abdomen sensor 116 and the chest sensor 102 may be placed in other areas of the abdomen 304 and chest 306, respectively, to account for different characteristics of each user 302. In optional embodiments, the abdomen sensor 116 and chest sensor 102 may also be placed directly on the skin of a user 302 or may be embedded in a layer of material 308. They may also be placed on top of the clothes of a user 302. In the current embodiment, when a user performs proper diaphragmatic breathing as it relates to their use scenario and as instructed by the control device 130 on which the interactive game is played, the chest sensor 102 will have minimal movement while the abdomen sensor 116 should move as much as possible up and down with each breath. The movement of the chest sensor 102 and abdomen sensor 116 will be further discussed in regard to FIGS. 4-5.

Still referring to FIG. 3, the abdomen sensor 116 and chest sensor 102 provide vibratory feedback when moving correctly or incorrectly through use of the vibrating motors (108 and 122). By way of example, if the chest sensor 102 is moving when it should not be, then the vibrating motor 108 would alert the user 302 to keep the chest sensor 102 still. In another example, if the user 302 is not moving the abdomen sensor 116 enough relative to the chest sensor 102, then the vibrating motor 108 can alert the user 302 to make an adjustment. In an optional embodiment, the abdomen sensor 116 and the chest sensor 102 may be operated without the use of the screen of the control device 130 by using the vibrating motors (108 and 122) to provide non-visual feedback to allow those individuals who are visually impaired to also utilize the device. In optional embodiments, the abdomen sensor 116 and chest sensor 102 may also be used to detect certain health conditions such as paradoxical breathing, which occurs when the diaphragm collapses during inhalation and expands during exhalation (the reverse of occurs during a normal breath).

While in one embodiment vibratory feedback may be used, in optional embodiments, auditory feedback in the form of verbal cues that emanate from the speakers of the control device 130 may be used. The auditory cues are configured to talk user's through techniques. As an example, a non-visual auditory feedback may comprise a series of beeps that rise and fall in tone with the rise and fall of the abdomen sensor 116. The tone may go off-key based on the movement of the chest sensor 102. If a user is playing the game well/correctly, they will hear the abdomen sensor 116 rise and fall in pitch and in key (not out of key). The greater the movement of the abdomen, the wider the range of tones the user may hear. Furthermore, auditory cues in the form of the real-time coaching may be employed via the speakers discussed with relation to FIG. 8.

Referring still to FIG. 3, one or more straps (310, 312, and 314) may be employed to securely hold the chest sensor 102 and the abdomen 116 in the proper place. In one embodiment, each strap may be adjustable in nature and be connected to either material 308, or to each sensor individually.

Referring now to FIGS. 4A-4B, exemplary sensor feedback graphs used with the diaphragmatic breathing system in accordance with one embodiment of the present system is presented generally at 400A and 400B. In one embodiment, the system may be provided with a base-line chart/graph 400A and an in-use chart/graph 400B. The base-line chart 400A is exemplary of the readings taken prior to the game being played. The in-use chart 400B is exemplary of the readings taken while the game is being played by the user. The system is further configured to utilize the communication interface, in a HIPPA compliant manner, to send the patient results to their Electronic Health Record (EHR) to be reviewed by their physician either concurrently or later tie as part of the patient's health record.

Still referring to FIGS. 4A-4B, in the current embodiment, the vertical axis 402 on both the base-line chart 400A and in-use chart 400B comprise a range of numbers from 0 to 100 and is used to represent the movement of the user's chest and abdomen with the accelerometers (112 and 126) located in the chest sensor 102 and the abdomen sensor 116, respectively. In one embodiment, the horizontal axis 408 of the base-line chart 400A and the in-use chart 400B each comprise two labels such as chest 404 and abdomen 406. In regard to FIG. 4A, the chest movement of the user measured by the chest sensor 102 is 50 and the abdomen movement measured by the abdomen sensor 116 is 50, which are taken prior to the game being played. While playing a game and performing the diaphragmatic breathing exercise, the reading will move above and below the calibrated point based upon the movement of the accelerometers (112 and 126), which are located in the chest sensor 102 and the abdomen sensor 116, respectively. In regard to FIG. 4B, an exemplary of a reading taken during performance of the diaphragmatic breathing game. The chest movement of the user measured by the chest sensor 102 is 50 and the abdomen movement measured by the abdomen sensor 116 is 100, which are taken while performing the game exercise and maintain the chest movement constant as he/she inhales. The abdomen movement of the user measured by the abdomen sensor 116 is 100, which indicates that the user 302 performs the game exercise as required and inhaled the maximum amount of air into the abdomen. As the user exhales its breath, the abdomen reading 406 should move towards 0. This raise/up and down movements of the chest reading 404 and the abdomen reading 406 will vary among users and will be required to be calibrated prior to each usage. In operation, the ability of a user to reach an accelerometer reading 402 of 100 or 0 will vary and become increasingly difficult as the game progresses, encouraging deeper diaphragmatic breaths as the game progresses.

Referring now to FIG. 5, an exemplary gameplay flow for use with the diaphragmatic breathing system in accordance with one embodiment of the present system is presented generally at 500. In one embodiment, the gameplay screen 502 is shown, which in this embodiment is a slingshot game. The gameplay screen 502 also comprises the accelerometer and/or gyroscope (though other sensors may be employed) feedback graph 504 as well as the instruction box 506. In this embodiment, the user 302 performs the game by using the instructions given in the instruction box 506. In optional embodiments, the instructions may be given by ways of audio via use of the speakers 148 on the control device 130. In one embodiment, the user 302 is instructed to keep the chest still while moving the belly during inhaling and exhaling in order to operate the slingshot and play the game. The better the user 302 is at complying with the instructions, the better score it will receive once it activates the slingshot. Furthermore, the optimization engine 168 collects all of the data regarding the user's performance via the assessment module 138. The optimization engine 168, via the machine learning module 164, is configured to make real-time adjustments to the interactive games based on the user performance, medical practitioner input, and/or both.

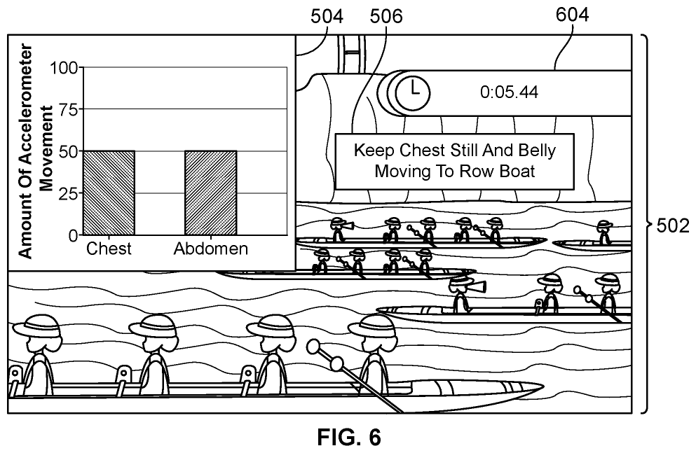

Referring now to FIG. 6, an exemplary gameplay flow for use with the diaphragmatic breathing system in accordance with one embodiment of the present system is presented generally at 600. In one embodiment, the gameplay screen 602 is shown, which is a rowing game. The gameplay screen 602 also comprises the accelerometer feedback graph 604 and the instruction box 606 and a timer 608. In this embodiment, the user 302 performs the game according to the instructions given in the instruction box 606. In optional embodiments, the instructions may be given by ways of audio via the speakers 148 of the control device 130. In one embodiment, the user 302 is instructed to keep the chest still while moving the belly during inhaling and exhaling in order to row the boat and play the game. Each combination of an inhale and exhale is one rowing stroke. The better the user 302 is at complying with the instructions, the faster the boat will row and the better the score will be once the timer ends.

With reference to FIGS. 5-6, the current embodiments presented only includes one user 302. However, in optional embodiments, multiple users may play the same game at the same time in order to compete against each other or against their old performances. This may be achieved through the use of the communication interface 154 located on each control device 130. By fostering a more competitive environment, users will be required to perform better in the diaphragmatic breathing games, thereby increasing the effectiveness and efficiency of the therapy. In optional embodiments, the games may be designed for specific types of users to encourage development of certain muscles. For example, a game is designed specifically for vocal performers as proper diaphragmatic breathing can allow singers to develop an increased lunch capacity and experience less tension while performing. The optimization engine, via the machine learning module 164, is configured to make real-time adjustments to the interactive games based on the user performance, medical practitioner input, and/or both. Examples of machine learning that may be employed are, but not limited to, neural networks, convolution neural networks, Random Forest (RF Tree 166), which is discussed in greater detail with reference to FIG. 7.

With reference now to FIG. 7, an example of a machine learning process for optimizing the user diaphragmatic breathing routines in real-time is presented. In one embodiment, random forests or random decision forests 166 are used to optimize breathing techniques. An RF ensemble learning method for classification, regression, and other tasks operates by constructing a multitude of decision trees at training time and outputting the class that is the mode of the classes (classification) or mean prediction (regression) of the individual trees. Random decision forests correct for decision trees' habit of overfitting to their training set and are trained independently by recursive binary partitioning of a bootstrapped sample of the input data, X. As shown in FIG. 7, the training set data 702 is generated. The training set data 702 comprises output data related to abdomen movement 704 and the chest movement 706, which are received from the chest sensor 102 and the abdomen sensor 116, and data from the user field 708 that is drawn from user data and population data base 722. Once the training set 702 is gathered data samples (710, 712, and 714), from those data samples, the system may generate one or more trees (716, 718 and 720).

Once the trees (716, 718 and 720) are generated, the user test set 728 for a single user during a routine, for example, is dropped down through each tree and the response estimate is the average over the all the individual predictions in the forest. Then using the trees (716, 718 and 720), a vote 724 occurs, which is configured to predict the probability of the user performing a proper breathing and then passes the information to the optimizing engine for optimization 736, which tunes the game accordingly. Furthermore, the historical performance data from users or a group of uses for generating and training the decision trees (e.g., the “training dataset). In operation, an example of a random forest approach to automatically modify the exercise, which is provided. In operation, each routine may be given a score by the user based on pain reduction effect to establish training data for the random forest. The medical professional may then group certain kinds of breathing games based on certain scores, and other various metadata such as, but not limited to, user age, gender, weight, and the like. Once the training data set 702 is established, a ranking depending upon the user input is developed. Once the trees (716, 718, and 720) are generated, the user performance data 326 (for a single user during a routine, for example, is dropped down through each tree and the response estimate is average over the all the individual predictions in the forest).

More details about the RFA may be found in L. Breiman, “Random Forests,” Machine Learning 45 (1):5-32 (2001) and A. Liaw et al., “Classification and Regression by Random Forest,” R News, Vol. 2/3, p. 18 (2002), both of which are incorporated by reference. In the typical instance, the RFA either a will identify one or more datasets based on posted media and make a standard assumption based on certain data features or they performance indicators (in this case provided by patients). The system data-mines such datasets, taking into consideration the specific patient attributes to extract a sufficient data within a specific category to train one or more deep learning algorithms.

In exemplary embodiments, a method for optimizing diaphragmatic breathing in an individual is presented. At one step, the first sensor is located or placed at a user's abdomen and the second sensor is located at user's chest to measure breathing movement of the abdomen and the chest. At another step, a third sensor is disposed at the user's mouth to measure an amount of air exhaled by the user, wherein third sensor is a mouthpiece. At another step, an upward breathing movement of the first sensor is detected via absence of airflow in the mouthpiece and downward breathing movement of the first sensor is also detected via air exiting through the mouthpiece. At another step, the measured breathing movement is utilized by a control device to measure user breathing and compared the user breathing with a predefined value. Further, at another step, the feedback is provided to the user to optimize diaphragmatic breathing.

In one embodiment, the step of outputting further comprises, at one step, one or more therapy games are provided for the user, which are controllable by using diaphragmatic breathing. At another step, performance data related to each therapy game played by the user is stored. Further, at another step, real time adjustments to the one or more therapy games are made based on the performance data to optimize diaphragmatic breathing.

Referring now to FIG. 8, a sensor strip 806 a vibrating device 802 and an audio device or a speaker 804 is disclosed. In one embodiment, the vibrating device 802 and the audio device 804 are securely and operably affixed to the sensor strip. In one embodiment, the vibrating device 802 may be a vibrating motor. The vibrating device/vibrating motor 802 is configured to provide alerts for the user 302 by providing vibratory feedbacks. In an exemplary embodiment, the vibrating device 802 is configured to alert the user 302 to still keep the chest sensor 102. In one exemplary embodiment, the vibrating device/vibrating motor 802 may alert the user 302 when the movement of the abdomen sensor 116 is not enough relative to the movement of the chest sensor 102 so that the user 302 may make adjustments.

In one embodiment, the sensor strip 806 further comprises one or more audio device or speaker 804. The audio device 804 is configured to provide audio alerts for the user 302. In one embodiment, the audio alerts may be voice alerts that includes vocal cues. In an exemplary embodiment, the audio device 804 is configured to alert the user 302 to still keep the chest sensor 102. In operation, a user may receive audio feedback from a smartphone describing proper techniques or making sounds that are indicative of a proper technique (e.g., waves). In one exemplary embodiment, the audio device 804 may alert the user 302 when the movement of the abdomen sensor 116 is not enough relative to the movement of the chest sensor 102 so that the user 302 may make adjustments. In one embodiment, the vibrating device 802 and the audio device 804 are powered using a power source, for example, a battery (110 and 124).

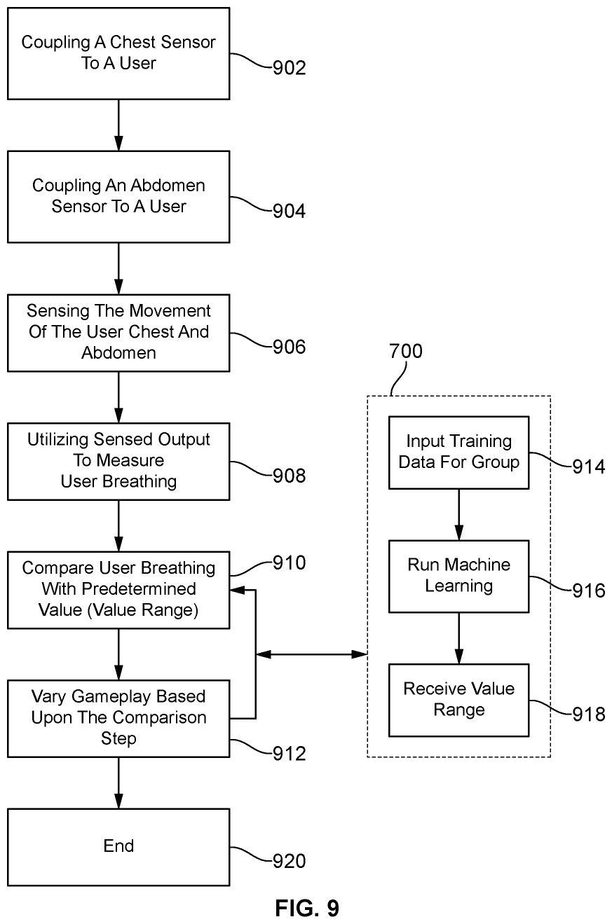

Referring to FIG. 9, a method for optimizing the diaphragmatic breathing or respirations is disclosed. At step 902, the user 302 couples or places the chest sensor 102 at the user's chest. At step 904, the user 302 couples or places abdomen sensor 116 at the user's abdomen. At step 906, the chest sensor 102 and the abdomen sensor 116 detect or sense the movement of the user's chest and abdomen, respectively. At step 908, the breathing or respirations of the user is measured by utilizing the sensed output from the chest sensor 102 and the abdomen sensor 116. At step 910, the respirations or breathings of the user is compared with the predetermined values (or a value range) based on the user or the user's condition or the user's goals. At step 912, gameplay is varied based upon the comparison of the respirations or breathings of the user with the predetermined values. At step 914, an input training data for a group may be received. At step 916, a plurality of machine learning programs may be run using the input training data. In one embodiment, the machine learning process is used for optimizing the user diaphragmatic breathing routines or respirations in real-time. At step 918, a value range is received from the machine learning program to optimize the system for a user-specific game. At step 920, the method for optimizing the user's diaphragmatic breathing or respirations is ended

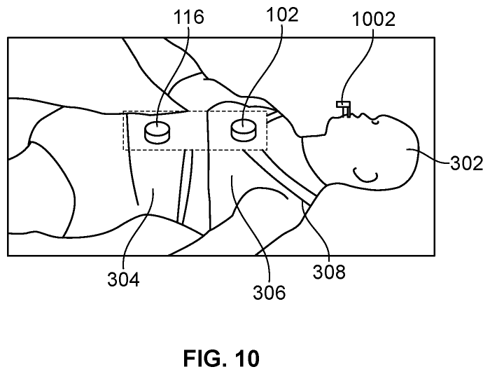

Referring now to FIG. 10, a third sensor 1002 is used together with the chest sensor 102 and the abdomen sensor 116 by the user 302 is disclosed. In one embodiment, the third sensor 1002 is configured to wirelessly communicate to the control device 130 using a wireless technology, for example, Bluetooth® technology. In one embodiment, the third sensor 1002 may be, but is not limited to, a mouth sensor, a mouthpiece air sensor and a Bluetooth mouthpiece. The third sensor 1002 is further configured to measure the amount of air being exhaled by the user 302. The movement of the abdomen sensor 116 with no air entering into the third sensor 1002 and the abdomen sensor 116 comes back down with air exiting via the third sensor 1002 may be detected using the third sensor 1002. In one embodiment, the chest sensor 102 may be directly placed on the user's chest 306 and the abdomen sensor 116 may be directly placed over the user's abdomen or stomach 304. In another embodiment, the chest sensor 102 and the abdomen sensor 116 are securely embedded in a layer of a material, for example, straps 308.

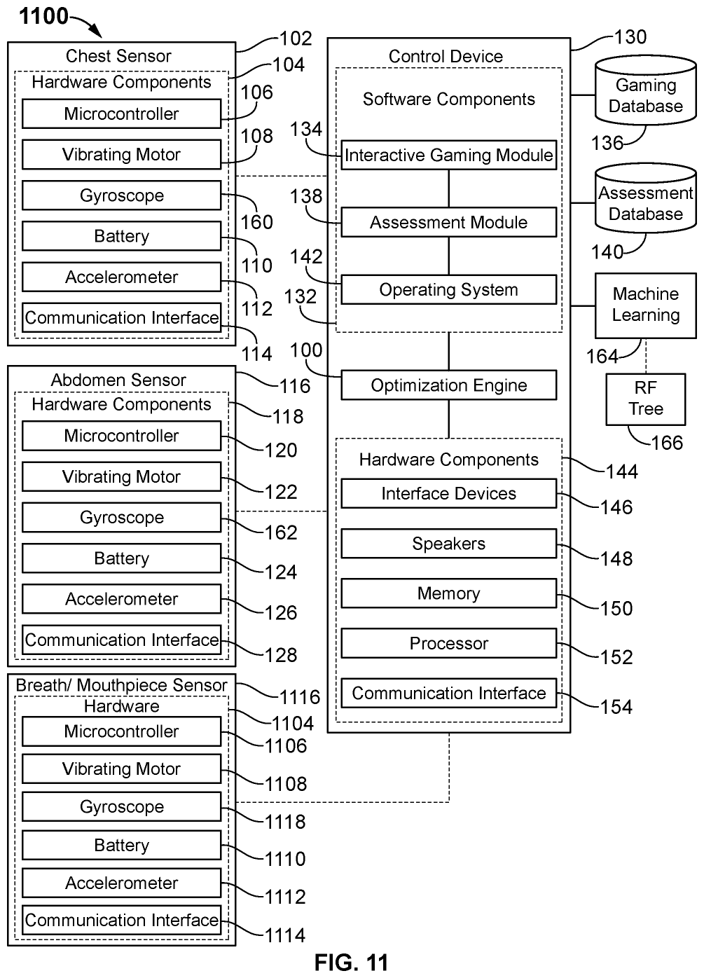

Referring now to FIG. 11, an exemplary diaphragmatic breathing system 1100 in accordance with another embodiment of the present system is disclosed. In one embodiment, the system 100 comprises the chest sensor 102, the abdomen sensor 116, the control device 130, and a breath/mouthpiece sensor 1116. In one embodiment, the mouthpiece sensor 1116 comprises a plurality of hardware components 1102. In one embodiment, the plurality of hardware components 1102 includes, but not limited to, a microcontroller 1106, a vibrating motor 1108, a power source 1110, an accelerometer 1112, a gyroscope 1118, and a communication interface 1114. In one embodiment, the mouthpiece sensor 1116 is configured to wirelessly communicate to, but not limited to, the control device 130 and any other remote computing device via the communication interface 1114. In one embodiment, the communication interface 1114 comprises, but not limited to, hardwired and wireless interfaces through which the mouthpiece sensor 1116 may wirelessly communicate to, but not limited to, the other devices, a network, and a plurality of servers. In an exemplary embodiment, the communication interface 114 comprises a Bluetooth® module and an antenna, which allows the mouthpiece sensor 1116 to wirelessly communicate with, but not limited to, the control device 130 and other systems. In one embodiment, the control device 130 may also be a portable device, such as, but not limited to, a tablet, a laptop computer, a netbook computer, a personal digital assistant (PDA), a mobile telephone, or other mobile device, and other communication devices. In optional embodiments, the communication interface 1116 may utilize other wireless technology or even wired technology depending on the usage including, but not limited to, Wi-Fi, long-term evaluation (LTE), global system for mobile communications (GSM), enhanced data rates for GSM evolution (EDGE), code-division multiple access (CDMA), worldwide interoperability for microwave access (WiMAX), and near-field communication (NFC), etc.

In one embodiment, the power source 1110 may be, but not limited to, a battery and a rechargeable lithium ion battery. In one embodiment, the accelerometer 1112 is configured to sense the movements along respective X, Y, and Z axes. In one embodiment, the microcontroller 1106 is configured to operate and control the vibrating motor 1108, the power source 1110, the accelerometer 1112, and the communication interface 1114. In one embodiment, the microcontroller 1106 is a small computer on a single integrated circuit chip, and comprises one or more central processing units (CPUs) along with a memory and the programable input and output peripherals. In some embodiments, a different computing device may be used to operate the vibrating motors 1108, the power source 1110, the accelerometer 1112, and the communication interface 1114. In operation, each of the hardware components 1104 of the mouthpiece sensor 1116 are configured to assess breathing inhaled and exhaled by the user, and use the data may be used as inputs to a machine learning algorithm for optimizing the user diaphragmatic breathing. In one embodiment, the mouthpiece sensor 1116 further comprises a gyroscope 1118. In one embodiment, the gyroscope 1118 is configured to sense or measure orientation and angular velocity or a change in rotation, which is unaffected by the acceleration.

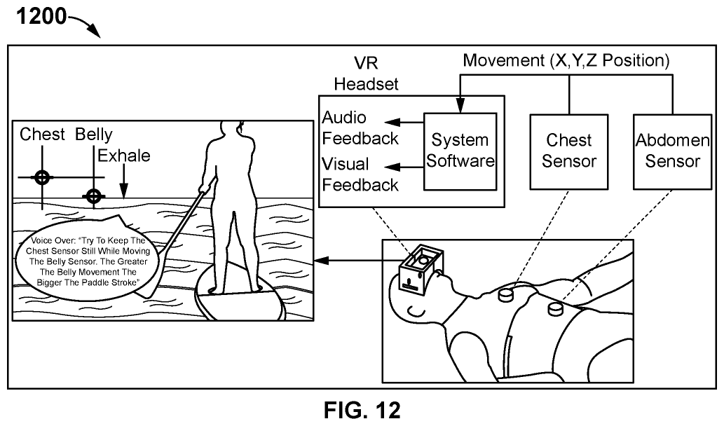

Referring to FIG. 12, a screen shot 1200 of a virtual reality headset used together with the chest sensor 102 and the abdomen sensor 116 by the user 302 is disclosed. In one embodiment, the VR headset is configured to communicate to the system for receiving inputs, thereby proving an audio feedback and visual feedback for the user 302. In one embodiment, the system may generate audio feedbacks and video feedbacks based on the data received from the chest sensor 102 and the abdomen sensor 116 and send to the VR headset while using. In one embodiment, the system may alert the user 302 via the VR headset. In an exemplary embodiment, a paddle surfing game is designed for different users. In an exemplary embodiment, the system may provide guidance for the user 302 while playing the paddle surfing via the VR headset. In an exemplary embodiment, if the user wants to stroke the paddle, then the system may provide a voice feedback such as try to keep the chest sensor 102 still while moving the abdomen sensor 116. In one embodiment, the user may effectively play the game by moving the belly or abdomen based on the instructions or feedback given by the system, thereby increasing the effectiveness and efficiency of the diaphragmatic breathing using the system.

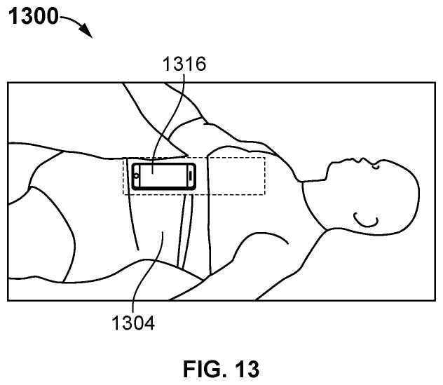

Referring now to FIG. 13, is a perspective view diagram 1300 of a smartphone 1316 being used as the sensing apparatus in accordance with one embodiment of the present system is presented. Smartphones 1316, as generally made or manufactured, comprise a microcontroller, a vibrating motor, an accelerometer, a gyroscope, and a communication interface. In this case, the smartphone 1316 is utilized as an abdominal sensor. However, it may be also utilized as a chest sensor if it is placed on the chest of the user within the dashed lines as shown. Furthermore, the smartphone 1316 may used in conjunction with a chest sensor for abdominal sensor, and it may be used alone as either an abdominal sensor or a chest sensor 1302 to sense motion and convert the inputs into breathing parameters.

The smartphone 1316 comprises a communication interface which may communicate with other devices, a network, and a plurality of servers and communicate via a downloadable mobile application. In the current embodiment, the communication interface may comprise a Bluetooth® module that allows the smartphone 1316 to communicate wirelessly with the control device and other system components at short range.

The accelerometer of the smartphone is configured to sense movement along the X, Y, and Z axis. The gyroscope of the smartphone is configured to sense a change in rotation which is unaffected by acceleration. In this way, in operation, each of the hardware and software components of the smartphone 1316 are configured to assess breathing movements of the chest or abdominal core, and use the data outputs to plot a breathing graph, and use the data outputs as inputs to a machine learning algorithm discussed above and to teach and train proper breathing techniques as discussed above. The vibratory motor or the speaker may provide tactile or audio feedback as described with relation the previous Figures.

In optional embodiments, the smartphone may also be placed on the abdominal rather than the chest, and used in conjunction with another sensing device. In other words, the smartphone 1316 may be used together with chest sensor 1302 or abdominal sensor 116, and the phone then acts as the chest sensor. In this way, user would only purchase a single sensor that communicates with the smartphone and the other components.

While the present system has been described in connection with what are presently considered to be the most practical and preferred embodiments, it is to be understood that the present system is not limited to these herein disclosed embodiments. Rather, the present system is intended to comprise the various modifications and equivalent arrangements included within the spirit and scope of the appended claims.

Although specific features of various embodiments of the system may be shown in some drawings and not in others, this is for convenience only. In accordance with the principles of the invention, system, the feature(s) of one drawing may be combined with any or all of the features in any of the other drawings. The words “including,” “comprising,” “having,” and “with” as used herein are to be interpreted broadly and comprehensively, and are not limited to any physical interconnection. Moreover, any embodiments disclosed herein are not to be interpreted as the only possible embodiments. Rather, modifications and other embodiments are intended to be included within the scope of the appended claims.

We claim:

1. A system to optimize diaphragmatic breathing, the system comprising:
a first sensor for measuring breathing movement of a user's abdomen and for outputting a signal related to the movement of the user's abdomen;
a second sensor for measuring breathing movement of the user's chest and for outputting a signal related to the movement of the user's chest; and
a control device communicatively coupled with the first sensor and the second sensor, wherein the control device comprises:
one or more processors;
a memory comprising a set of program modules executable by one or more processors;
an assessment module for receiving the signals from the first sensor and the second sensor and converting the signals to a data input, and further, for comparing the data input to a predetermined data range representative of proper diaphragmatic breathing for the user; and
a communication interface in communication with the assessment module, the communication interface utilized to provide feedback to the user based on the assessment modules comparison of the data input and the predetermined range so as to optimize the user's diaphragmatic breathing.

2. The system of claim 1, wherein the feedback comprises audio feedback, visual feedback, tactile feedback, or any combination thereof, and wherein the first sensor or the second sensor is a smartphone having an accelerometer, gyroscope, or both.

3. The system of claim 1, further comprises a vibrating device disposed within the first sensor, second sensor, or both, wherein the control device signals the vibrating device to provide vibratory feedback to signal the user to adjust breathing movement in the chest, abdomen, or both.

4. The system of claim 1, wherein the control device comprises:
an interactive gaming module in communication with a gaming platform comprising one or more therapy games, wherein the interactive gaming module enables a user to control one or more therapy games for training the user as to proper diaphragmatic breathing;
an optimization engine in communication with the assessment module for making real-time adjustments to the one or more therapy games based on a performance data captured to optimize diaphragmatic breathing;
wherein the assessment module in communication with an assessment database, wherein the assessment database stores the performance data related to each therapy game played by the user;
wherein the database is in communication with an Electronic Health Record (EHR) in a HIPPA complaint manner such that the performance may be reviewed by a medical professional.

5. The system of claim 4, wherein the one or more processors is implemented with a machine learning algorithm, and the machine learning algorithm, when executed, is configured to:
receive training data from a data source;
utilize the training data make real-time adjustments to the interactive games based on the user's performance, a medical professional input, or both.

6. The system of claim 5, wherein the machine learning algorithm is random forest.

7. The system of claim 1, wherein the control device is a computing device, a mobile device, a wearable device, or a wearable digital headset device in the form or a virtual reality or augmented reality headset.

8. The system of claim 1, further comprising a third sensor for measuring an amount of air exhaled by the user's mouth and output a signal related to amount of air exhaled through the user's mouth, wherein the third sensor is communicatively coupled to the control device, wherein the third sensor is a mouthpiece.

9. The system of claim 1, further comprising a fourth sensor for measuring an amount of air flow from a nasal passage of the user, wherein the fourth sensor is communicatively coupled to the control device.

10. The system of claim 1, wherein each of the first, second and third sensors comprise at least one of:
an accelerometer to sense movement along the X, Y, and Z axis; and
a gyroscope for to sense a change in rotation which is unaffected by acceleration.

11. A method to optimize diaphragmatic breathing, the method comprising:
locating a first sensor proximate a user's abdomen;
locating a second sensor proximate a user's chest;
capturing, using the first and second sensor, movement of the chest and abdomen during user diaphragmatic breathing;
outputting a signal based on the movement of the chest and abdomen to a control device via a communication protocol;
receiving the signal from the first sensor and second sensor and converting the signals to a data input via an assessment module in communication with a processor;
comparing, using a processor, the data input to a predetermined data range representative of proper diaphragmatic breathing for the user; and
providing feedback to the user based on the assessment modules comparison of the data input and the predetermined range so as to optimize the user's diaphragmatic breathing.

12. The method of claim 11, wherein the feedback comprises audio feedback, visual feedback, or tactile feedback, or any combination thereof.

13. The method of claim 12, wherein providing tactile feedback comprises providing a vibrating device disposed within the first sensor, second sensor, or both, wherein the control device is configured to provide vibratory feedback via the first sensor, second sensor, or both, to signal the user to adjust breathing movement in the chest, abdomen, or both.

14. The method of claim 11, further comprising:
connecting the user, via a network, to an interactive gaming module in communication with a gaming platform comprising one or more therapy games;
enabling the user to control one or more therapy games for training the user as to proper diaphragmatic breathing;
receiving, via the processor, performance data from the gaming module;
during game play, adjusting the one or more therapy games in real time based on received performance data to optimize diaphragmatic breathing;
storing, in a database, performance data related to each therapy game played by the user.

15. The method of claim 14, further comprising:
executing a machine learning algorithm;
receiving training data from a data source;
utilizing the training data make real-time adjustments to the interactive games based on the user's performance, medical practitioner input, or both.

16. The method of claim 11, wherein the control device is a computing device, a mobile device, a wearable device, or a wearable digital headset device in the form or a virtual reality or augmented reality headset.

17. The method of claim 11, further comprising:
locating a third sensor proximate the user's mouth;
capturing, using the third sensor, an amount of air exhaled by the user's mouth and output a signal related to amount of air exhaled through the user's mouth;
wherein the third sensor is a mouthpiece;
wherein the sensor is configured to detect an upward movement of the first sensor via absence of airflow in the mouthpiece, and detect a downward movement of the first sensor via air exiting through the mouth;
locating a fourth sensor proximate the user's nasal passage,
capturing, using the fourth sensor, an amount of air flow from a nasal passage of the user;
outputting the captured signals of the third and fourth sensor to the control device.

18. A system to optimize diaphragmatic breathing, the system comprising:
a smartphone having an accelerometer, gyroscope, or both disposed therein, the smartphone being utilized to collect data regarding movement of a user's chest, abdomen, or both, during diaphragmatic breathing exercises when the smartphone is placed on the chest or abdomen of the user;
an application on the smartphone to receive an output signal related to the movement of the user's abdomen;
wherein the mobile application comprises:
an assessment module for receiving the signal from the first sensor and second sensor and converting the signals to a data input, and for comparing the data input to a predetermined data range representative of proper diaphragmatic breathing for the user; and
a communication interface for providing feedback based on the assessment modules comparison of the data input and the predetermined range so as to optimize the user's diaphragmatic breathing.

19. The system of claim 18, wherein the feedback comprises audio feedback, visual feedback, or tactile feedback, or any combination thereof.

20. The system of claim 1, wherein the application signals the smartphones vibration module disposed within the smartphone to provide vibratory feedback to signal the user to adjust breathing movement in the chest, abdomen, or both.
